(19) United States
(12) Patent Application Publication
(10) Pub. No.: US 2003/0144359 A1
(43) Pub. Date: Jul. 31, 2003

(54) Dermatological use and a dermatological preparation

(76) Inventor: Pekka Heino, Helsinki (FI)

Correspondence Address: JACOBSON HOLMAN PLLC, 400 SEVENTH STREET N.W., SUITE 600, WASHINGTON, DC, 20004, US

(21) Appl. No.: 10/239,882

(22) PCT Filed: Feb. 5, 2001
(86) PCT No.: PCT/FI01/00100

(30) Foreign Application Priority Data
Apr. 6, 2000 (FI) 2000818

(51) Int. Cl. A61K 31/198

(52) U.S. Cl. 514/567

(57) ABSTRACT

The present invention concerns a dermatological use, a dermatological preparation and a method of treatment to inhibit or reduce the thyroid hormone activity of L-thyroxine and its metabolite L-triiodothyronine, which accelerate cellular metabolism and proliferation. A topical pharmaceutical preparation containing a thyronine derivative inhibiting the thyroid hormone activity of L-thyroxine and L-triiodothyronine is used.

This invention relates to a dermatological use, a dermatological preparation and a treatment with the same. In this invention it is treated of proliferative skin conditions, i.e. conditions manifested as accelerated multiplication of cells and as therewith associated disorders of cellular growth and differentiation.

The thyroid gland synthesizes and secretes thyroid hormones, which are iodine-containing derivatives of the amino acid thyronine. These hormones exhibit a biological activity known as thyroid hormone activity. The various compounds differ in their degree of thyroid hormone activity. Physiological and synthetic compounds possessing such activity are collectively known as thyroid hormone analogues. Hundreds of such compounds are known.

The most well-known physiological thyroid hormone analogue is L-thyroxine, or 3,3′,5,5′-tetraiodo-L-thyronine (T4), with four iodine atoms bound to the thyronine skeleton

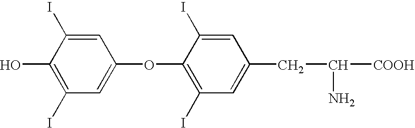

It is an essential human hormone regulating the general rate of metabolism, as well as activating cell proliferation (their multiplication, growth and differentiation). Abnormal increases in the amount of thyroxine, as in hyperthyroidism, cause acceleration of cellular and tissue metabolism, manifested clinically as thyroid hormone poisoning, or thyrotoxicosis. The symptoms of this hyper metabolic syndrome are also evident on the skin. In patients with psoriasis, for instance, the patient's disease typically worsens. T4 is mostly metabolized through de-iodination in the liver and peripheral tissues, including the skin, to L-triuodothyronine; 3,3′,5-triiodo-L-thyronine, T3, containing three iodine atoms.

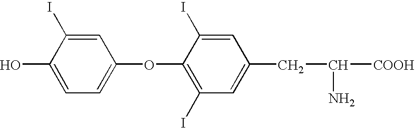

T3 has a thyroid hormone activity five times that of T4.

Some of the T4 is metabolized to 3,3′,5,5′-tetraiodothyropropionic acid, T4P,

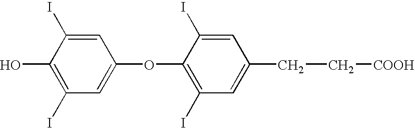

through deamination of the side chain of the thyronine skeleton or to 3,3′,5,5′-tetraiodothyroacetic acid, T4A,

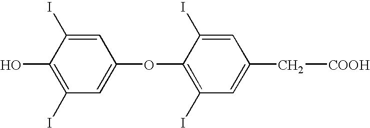

through side chain deamination and shortening. These tetraiodothyrocarboxylic acids possess only about one-fourth of the thyroid hormone activity of their parent compound, T4.

Patients with untreated hypothyroidism exhibit significant skin symptoms in addition to other organ symptoms related to the reduced metabolic rate caused by T4 (T3) deficiency. Acid glycosaminoglycans accumulate in the subdermal layer, and connective tissue fibers are reduced in quantity and changed in quality. The skin becomes cold, yellowish and dry. Its cornified outer layer, the epidermis, becomes thick and coarse. The elbows and knees especially may develop coarse, dirty brown hyperkeratosis, i.e. epidermal thickening, also known as the “dirty knee symptom”.

Systemic administration of T4 (or T3) quickly removes these symptoms and normalizes the skin.

Of the thyroid hormone analogues, only T4 and T3 are used in therapeutic practice, with one or the other being administered internally as hormone substitution therapy for hypothyroidism-D-Thyroxine, i.e. the D-isomer of thyroxine, which has substantially less thyroid hormone activity than T4, has been tested as an agent to reduce blood lipid levels (Farwell A P, Braverman L E (1996): Thyroid and Antithyroid Drugs. In Goodman & Gilman's The Pharmacological Basis of Therapeutics, 9th ed Eds. Hardman J G, Limbird L E, Molinoff P B, Rudden P W, Goodman Gilman A. McGraw-Hill, New York, p. 1383-1409).

In addition to internal administration, prior art also includes topical administration of thyroid hormone analogues (e.g. NZ 207923, U.S. Pat. No. 5,856,359, 5,951,989, WO 9640048). Topical administration has been undertaken to reduce the systemic side effects caused by the compounds' thyroid hormone activity and to find new indications for their use.

The present invention uses thyronine derivatives (e.g. T4A and T4P) possessing a weaker thyroid hormone activity than T4 to pharmacologically inhibit or reduce the thyroid hormone activity of T4 (and T3). When T4A and/or T4P, both of which have weak thyroid hormone activity compared with that of T4 or T3, are/is applied topically to a target area in sufficient amount(s), local hypothyroidism is produced in this area. By utilizing this phenomenon, appreciable new therapeutic benefits can be achieved in proliferative dermatological conditions where an absolute or relative local excess of T4 (and T3) constitutes the etiological and/or disease-maintaining factor. This pertains not only to situations of systemic elevation of T4/T3 levels (thyrotoxicosis) but also to situations where a tissue area reacts locally to the hypermetabolic effect of T4/T3 (as in psoriasis, see below).

In consequence of the above, the present invention and the compounds defined in it are not aimed at achieving clinical and/or therapeutic effects in conditions characterized by reduced concentrations of T4/T3 in the target tissue. These include the dermal manifestations of hypothyroidism, where there is an absolute reduction in T4/T3, and also skin conditions where the tissue response to T4/T3 is reduced, i.e. where a relative deficiency of these hormones is the etiological factor.

The present invention is intended to be applied only in proliferative skin diseases, i.e. in conditions where the multiplication and/or growth of dermal cells is pathologically accelerated and/or their differentiation is deficient wholly or partly because of the sensitization of these cells to the effects of T4 and T3.

One example of such diseases is psoriasis. It manifests as scaly plaques on the skin, mostly on distal parts of the body such as elbows, knees, scalp and fingers. The lesions have an erythematous base covered by a thick layer of glossy, greasy, silvery grey scales. The psoriatic plaques vary in size, shape and number but they are always sharply demarcated from healthy skin. While the epidermal layer of healthy skin regenerates in about six weeks, cell production is elevated up to tenfold in psoriatic skin. Cells do not have the time to keratinise normally and, as a result, they are shed from psoriatic plaques in the form of characteristic scales.

The most practicable drug for topical treatment of mild or moderate psoriasis has until now been calcipotriol, a derivative of vitamin D. Its available pharmaceutical forms for topical therapy include ointments, creams and cutaneous solutions. The full effect of calcipotriol therapy is seen after 6 to 8 eight weeks of treatment. Still, complete removal of dermatitic plaques is achieved in only about 15% of cases (Guzzo C A, Lazarus G S, Werth V P (1996): Dermatological Pharmacology. In Goodman & Gilman's The Pharmacological Basis of Therapeutics, 9th ed. Eds. Hardman J G, Limbird L E, Molinoff P B, Rudden P W, Goodman Gilman A. McGraw-Hill, New York, p. 1593-1616).

When the present invention is utilized and topical pharmaceutical preparations containing the thyronine derivatives defined in the present invention are applied to psoriatic plaques, for instance, the therapeutic effect on the plaques is faster and more potent than even that produced by topical calcipotriol therapy.

Patent document WO 9640048, which bears a resemblance to the present invention, describes the topical use of thyroid hormone analogues in a group of proliferative and nonproliferative skin conditions. It is evident from the description and the associated exemplifying embodiments that the principle of said invention is not based on inhibiting or reducing the thyroid hormone effect of T4 and T3, i.e. actions characteristic of the present invention. That WO 9840048 is a distinct invention is indicated, firstly, by the fact that the effect of the invention is demonstrated by means of a dermal tissue model in which the pursued thyroid hormone activity is completely independent of the effects or presence of T4 or T3. Further, apart from the fact that a subsequent exemplifying embodiment of said invention mentions a beneficial effect on psoriasis from a topically administered thyroid hormone analogue (triiodothyroacetic acid), the same invention allows equally well T4 or T3 to be used for topical treatment of psoriasis. And yet these thyroid hormone analogues have an exclusively harmful topical effect on this disease. The present invention removes this fundamental disadvantage not only in practice but also logically. Thus, the invention described in WO 960048 and the present invention differ essentially from each other and are therefore distinct inventions.

The invention is characterized by what is stated in the patent claims.

Use of the invention will be described in the following exemplifying embodiment:

### EXEMPLIFYING EMBODIMENT

Simultaneous topical treatment of four untreated, typically scaling and sharply demarcated dermatitic plaques (plaques 1-1) in a psoriatic patient was undertaken. Plaque 5 was left untreated for comparison.

The areas of the plaques at initiation of the treatment were as follows: plaque 1: 8.7 cm²; plaque 2: 8.1 cm²; plaque 3: 6.8 cm²; plaque 4: 6.1 cm²; plaque 5: 6.4 cm².

The following ointments were applied to plaques 1-4:

Plaque 1

Locobase® unctuous cream (Yamanouchi Europe B. V., Leiderup, Holland).

Plaque 2

Daivonex® calcipotriol ointment containing 50 μg calcipotriol per gram (Løvens Kemiske Fabrik, Ballerup, Denmark).

Plaque 3

Locobase®D unctuous cream containing 500 μg 3,3′,5,5′-tetraiodothyroacetic acid, T4A, per gram (University Pharmacy, Helsinki, Finland).

Plaque 4

Locobase® unctuous cream containing 500 μg T4A per gram and 50 μg L-thyroxine, T4, per gram (University Pharmacy, Helsinki, Finland).

Each ointment was applied to the plaques at 50 mg/cm² twice in 24 hours, corresponding to 5 μg calcipotriol per cm² per 24 hours for plaque 2, 50 μg T4A per cm² per 24 hours for plaques 3 and 4, and 5 μg T4 per cm² per 24 hours for plaque 4. Treatment was continued in this manner for 35 days.

The results are presented in Table 1 below, showing the change (reduction) in plaque area with time (days).

TABLE 1

| | 14 days | 21 days | 28 days | 35 days | |
|---|---|---|---|---|---|
| Plaque 1: | 0% | 0% | 0% | 0% | (Locobase ®) |
| Plaque 2: | 35% | 55% | 65% | 85% | (Daivonex ®) |
| Plaque 3: | 45% | 65% | 85% | 90% | (T4A) |
| Plaque 4: | 40% | 60% | 85% | 90% | (T4A + T4) |
| Plaque 5: | 0% | 0% | 0% | 0% | (no treatment) |

It can be seen from Table 1 that both the T4A ointment and the T4A+T4 ointment had a clear effect, and they were similar in therapeutic efficacy. Measured in terms of reduction in plaque area, their therapeutic efficacy was better than that of the calcipotriol ointment.

The therapeutic trial was continued from 35 days onward as follows:

Plaque 1

Locobase® ointment.

Plaque 2

No treatment.

Plaque 3

Locobase® ointment.

Plague 4

Locobase® ointment containing 50 μg L-thyroxine, T4, per gram (University Pharmacy, Helsinki, Finland).

Plaque 5

No treatment.

As previously, the ointments were applied to the target plaques at 50 mg/cm² twice in 24 hours. Thus, the dose of T4 to plaque 4 was 5 μg T4 per cm² per 24 hours.

The results are presented in Table 2 below.

TABLE 2

| | 42 days | 50 days | 62 days | 70 days | |
|---|---|---|---|---|---|
| Plaque 1: | 0% | 0% | 0% | 0% | (Locobase ®) |
| Plaque 2: | 85% | 80% | 80% | 70% | (no treatment) |
| Plaque 3: | 95% | 95% | 90% | 80% | (Locobase ®) |
| Plaque 4: | 70% | 25% | −10% | −50% | (T4) |
| Plaque 5: | 0% | 0% | 0% | 0% | (no treatment) |

It can be seen from Table 2 that although some rash did return over four weeks to those skin lesions on which the treatment with calcipotriol or T4A ointments had been discontinued, the T4 ointment in fact reinstated the rash completely to plaque 4 previously treated with T4A+T4 ointment. Indeed, the T4 ointment increased the area of plaque 4 by 50% compared with baseline (Table 1). Therefore, it can be concluded that psoriatic rash is worsened by topically applied T4 (T3) and that T4A completely inhibits this effect and is also therapeutically efficacious.

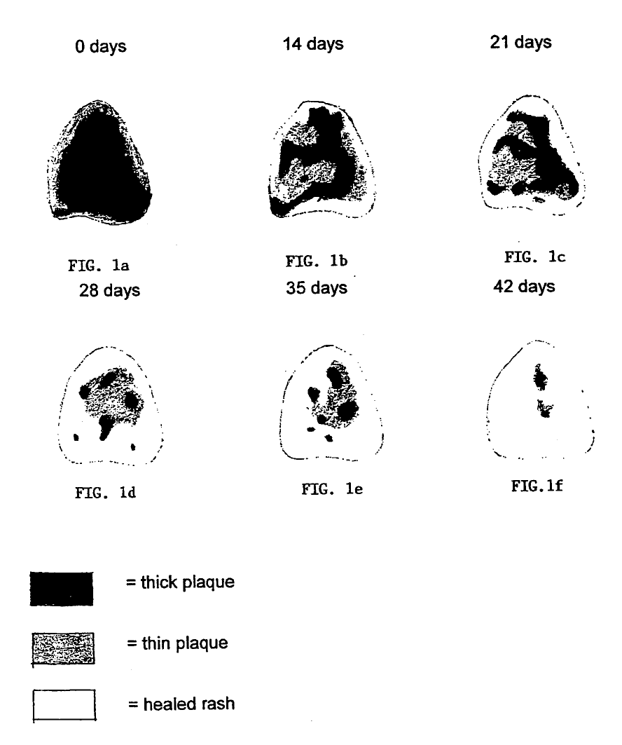

The topical effect of T4P, 3,3′,5,5′-tetraiodothyropropionic acid ointment on a previously untreated psoriatic plaque, on psoriasis was studied in a therapeutic design identical with the above. The dosage of T4P was 25 μg/cm² twice per 24 hours for 35 days. The follow-up period was 42 days. The ointment base was the same at the same concentration and dosage as T4A previously, T4P was found to completely inhibit the effects of T4 and to be equal to T4A in therapeutic efficacy (FIGS. 1a-1f). Effect of 3,3′,5,5′-tetraiodothyropropionic acid (T4P) ointment on a previously untreated psoriatic plaque. The dosage of T4P was 25 microg/cm² twice per 24 hours for 35 days. The follow-up period was 42 days.

1. Use of 3,3′,5.5′-tetraiadothyroacetic acid or 3,3′,5,5′-tetraiodothyropropionic acid, for preparing a topically administered medicinal preparation for treating proliferative skin diseases.

2. Use according to claim 1, wherein the proliferative skin disease is psoriasis.
